(19) United States
(12) Patent Application Publication
(10) Pub. No.: US 2018/0250351 A1
(43) Pub. Date: Sep. 6, 2018

(54) Cyanobacterial microalgae phycocyanin and phycocyanobilin to beneficially inhibit the activity of the UDP-GDH enzyme while significantly increasing the absorption and circulation of curcumin

(71) Applicant: Stefano SCOGLIO, Urbino (IT)

(72) Inventors: Stefano SCOGLIO, Urbino (IT); Gabriel Dylan SCOGLIO, Urbino (IT)

(21) Appl. No.: 15/757,354

(22) PCT Filed: Sep. 3, 2015
(86) PCT No.: PCT/EP2015/070102

§ 371 Date: Mar. 3, 2018

(51) Int. Cl. A61K 35/748

(52) U.S. Cl. CPC A61K 35/748; A61K 31/12; A61K 2300/00

(57) ABSTRACT

The present invention provides a method for inhibiting the UDP-GDH enzyme, which has in itself important beneficial implications, thus strongly enhancing the absorption and circulation of the natural drug curcumin, which in itself is poorly absorbed by the human organism. The method consists in blending curcumin with whole cyanobacterial algae, particularly Aphanizomenon flos aquae (but also Spirulina) or algal extracts concentrating or purifying the cyanobacterial molecules phycocyanin and phycocyanobilin. The method solves the significant problem of poor curcumin absorption through substances that also add their own nutritional and antioxidant activity to the mix.

### BACKGROUND OF THE INVENTION

Curcumin, the chromophore active principle of the common spice Turmeric (Curcuma longa), has long been a drug of choice for treating inflammatory conditions in Ayurvedic medicine. In the last 30 years numerous studies, both in vitro and in vivo on animals, have shown that curcumin possesses powerful antioxidant (1), antinflammatory (2) and anti-cancer (3) activity. A PubMed search on curcumin gives more than 6000 studies in the last 30 years; and now there quite a few clinical trials under way on human pathological conditions. As it has been written: “Based on early cell culture and animal research, clinical trials indicate curcumin may have potential as a therapeutic agent in diseases such as inflammatory bowel disease, pancreatitis, arthritis, and chronic anterior uveitis, as well as certain types of cancer.” (4) However, the therapeutic usefulness of curcumin has been shown to be severely limited by its very poor availability and absorption, and its rapid elimination from the body before it can reach any significant concentration and thus activity. (5) This has led to the search of substances capable of enhancing the absorption and circulation of curcumin, and thus its effectiveness, among them piperine (6) and phosphatidylcholine (7). However, besides their limited effect in absorption enhancement, these substances have other serious limitations: piperine, the active principle of black pepper, has potential side effects; while phosphatidylcholine, though devoid of side effects, has a lower absorption-enhancing power than piperine and in any case is a neutral substance which does not add any further activity to the blend. Cyanobacterial microalgae are organisms endowed with various nutritional and nutraceutical properties, and have been eaten as supplements for many decades now. In particular, two types of microalgae have been widely used, namely Spirulina, whose different species are artificially grown all over the world; and Aphanizomenon flos aquae, that grows spontaneously in Upper Klamath Lake, Oreg., USA, as well as in other lakes around the world, and that is now also being grown artificial environments. Both microalgae are characterized by a wide nutritional profile, and also contains numerous antioxidant molecules, including carotenoids and xanthophylls; chlorophyll and MAAs (mycosporine-like aminoacids); and unique cyanobacterial molecules, phycocyanins, that have proven to be the most powerful of the purified antioxidant molecules (8), and which in turn also contain the chromophore phycocyanobilin.

We have decided to focus our research on the Aphanizomenon flos aquae microalgae (AFA), mainly for its preferred lipid content, as it also contains relevant quantity of Omega 3 fatty acids. However, I have also then focused the research on the purified phycocyanins and phycocyanobilin, which have been purified from AFA but are for the most part common to all microalgae, and have then tested the substances in vivo, including also Spirulina. The use of a whole cyanobacterial algae, as well of the specific molecules contained in it and purified, has been shown to significantly increase the absorption of curcumin, while enhancing its functional health-supporting activity.

### DISCLOSURE OF THE INVENTION

Curcumin has been shown to be very effective and potentially very useful as a therapeutic tool in all pathologies involving oxidation and oxidative stress, inflammation, and cancer cell growth. The problem is that, once ingested alone, it is very poorly absorbed and utilized. The reasons for this are possibly many, but 3 have emerged as the main culprit: a) most of the curcumin ingested is blocked at the gut level, being unable to pass through the intestinal membrane, also due to the glucuronidation that already happens at the gut level; b) the curcumin that actually goes through the gut is then quickly metabolized by the liver through a process of liver glucuronidation, thus being made ready to be easily excreted from the body; c) whatever curcumin survives these two previous steps needs to be absorbed inside the cells, and again this seems to be a difficult process since cell walls constitute another significant barrier to absorption. The solutions that have been proposed to solve the problem of curcumin absorption have to do with solving one or more of the above mentioned problems. For instance, piperine has been shown to enhance curcumin absorption thanks to its ability to inhibit glucuronidation, thus preventing or slowing down significantly the process of excreting curcumin from the body, and therefore making curcumin more available. The way in which piperine does that is by its ability to inhibit the enzyme UDP-glucose dehydrogenase, the enzyme that catalyzes the conversion of curcumin into curcumin glucuronide.

UDP-glucose dehydrogenase is an enzyme belonging to the enzymes family of oxidoreductases, which catalyze reaction by way of oxidation. Up to a point, all anti-oxidant molecules can thus inhibit the UDP-glucose dehydrogenase, but only some such molecules have a special affinity for such enzyme. We thus have decided to test some of the known anti-oxidant molecules present in in cyanobacterial algae, as well as the whole AFA algae, for their ability to inhibit the UDP-glucose dehydrogenase. We have thus set as hypothesis to be tested that the cyanobacterial algae and the their anti-oxidant molecules may exert a powerful inhibitory activity over both the intestinal and the liver glucuronidation of curcumin, thus making it both more available in the passage from the gut to the liver, and then from the liver to the bloodstream. This, as we shall see, is exactly what happens when curcumin is ingested together with AFA algae as well as with Spirulina algae, so that the plausible mechanism of action we indicated above is thus confirmed both in a laboratory test and in practice. The inhibition of the UDP-GDH enzyme has beneficial properties of its own, as it counteracts the oxidating activity of the enzyme, and it also helps prevent its cancer-promoting activity (8).

The other way in which curcumin absorption and utilization is impaired, as we have seen, is its being blocked by the cell wall barrier. In relation to this, studies have shown that the blending of curcumin with lipids may enhance its ability to penetrate cell walls, precisely thanks to the affinity of lipids with the prevalent fatty component of cell membranes. This ability of liposoluble substance to enhance curcumin absorption is indeed relevant not only for the final incorporation of curcumin into the cells of organs and tissues, but also to its ability to penetrate the intestinal cell wall, and so to enter the path leading to the processing by the liver, where in any case fatty substances as such seem unable to prevent its glucuronidation. AFA algae emerges as relevant even in this respect, insofar as it has a significant content of fatty acids, which constitute approximately 5% of its dry weight, half of which being represented by essential polyunsaturated fatty acids. There is no doubt that this significant endowment of high quality fatty acids of AFA algae may further contribute to the increase of curcumin integral absorption into the bloodstream. Moreover, other liposoluble substances present in Klamath algae are liposoluble antioxidants such as the carotenoids, chlorophyll and vitamin E, all present in AFA algae. While Spirulina also contain some of these liposoluble molecules, its lipid profile is clearly inferior to that of AFA, and this is the reason why AFA is the preferred microalgae for the enhancement of curcumin absorption.

For all the reasons explained above, we have decided to proceed with two distinct and sequentially organized tests: a) a laboratory test to evaluate the ability to inhibit UDP-glucose dehydrogenase (UDP-GDH) of the whole AFA algae, as well as of the cyanobacterial molecules phycocyanins, and their pigment phycocyanobilin; b) a test on healthy human subjects to verify if the absorption of curcumin in the blood was enhanced by blending curcumin with the whole AFA algae, with an AFA extract concentrating phycocyanins and phycocyanobilin, and with Spirulina. We found that both tests gave positive results, confirming that mixing curcumin with cyanobacterial algae, as well as with specific microalgal molecules, significantly increased curcumin absorption relative to curcumin alone.

### DETAILED DESCRIPTION OF THE INVENTION

### 1) Laboratory Test on the Inhibition of the UDP-Glucose Dehydrogenase

The UDP-GDH enzyme catalyses the oxidation of the UDP-glucose using the NAD+ as the oxidant agent, according to the following formula:

UDP-glucose+2NAD++H2O→UDP-glucuronic acid+2NADH+2H+

For the spectrophotometric evaluation of the enzymatic activity the following reactive mix has been used:

| | |
|---|---|
| Tris-HCl | 100 mM pH 8.5 |
| UDP-glucose | 1 mM |
| NAD+ | 2 mM |
| UDP-GDH | 2.5 μg/ml (0.35 mU/ml) |

±Inhibiting agent: AFA algae water-soluble extract + ethanolic extract; phycocyanin, phycocyanobilin.

The test has been done at 37° C. and monitored at 340 nm for 20 minutes.

### Preparation of the Extracts

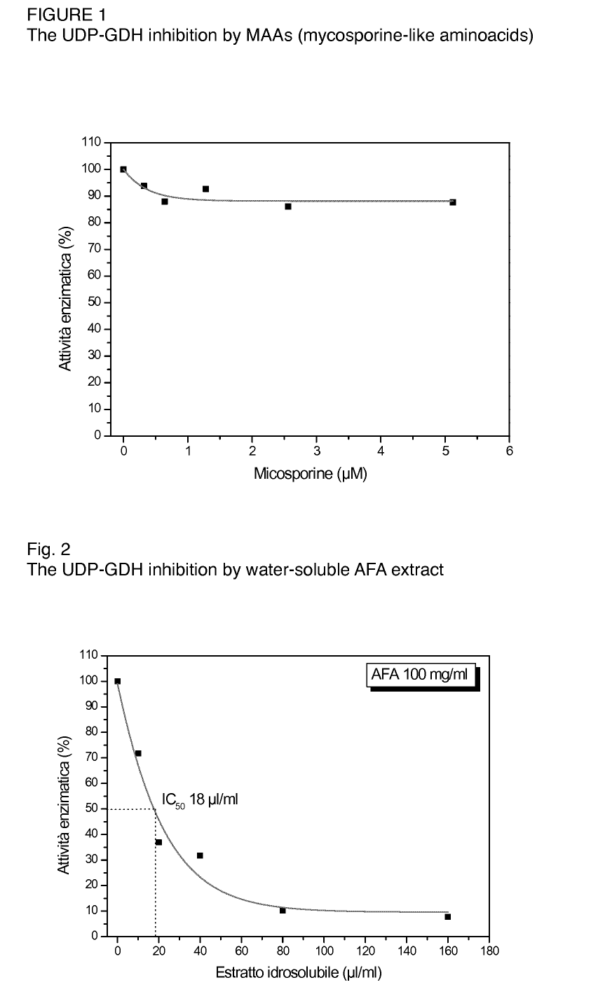

The reason why we have prepared the extracts is because we could not properly dissolve the whole algae in a single medium to prepare it for spectrophotometric analysis. We have thus decided to do two separate extracts, one water-soluble and the other lipid-soluble or ethanolic, to evaluate the UDP-GDH inhibiting properties of the two fractions of the algae, then making a median of the results to establish the inhibiting potential of the whole algae. Also since the MAAs (mycosporine like aminoacids) would interfere with the spectrophotometric analysis, we have removed the MAAs from the water-soluble extract through a dialysis, and tested them separately: we have found that their UDP-GDH inhibiting property is almost null (FIG. 1), so their presence in the whole algae does not add anything significant in relation to the inhibition of this enzyme do not add anything to the whole algae.

The algae has thus been suspended in different concentrations (10-50-100 mg/ml) in distilled water on the one hand and in 100% ethanol on the other; then homogenized and afterwards centrifuged at 5000 rpm for 10 minutes. We have then checked through spectrophotometry the presence in the two extracts of the relevant components, verifying the presence of phycocyanins at 620 nm for the water extract and of carotenoids at 400 nm and chlorophyll at 664 nm for the ethanol extract.

Finally, phycocyanins and their chromophore phycocyanins have been purified for their independent evaluation following known methodologies. (9)

Results

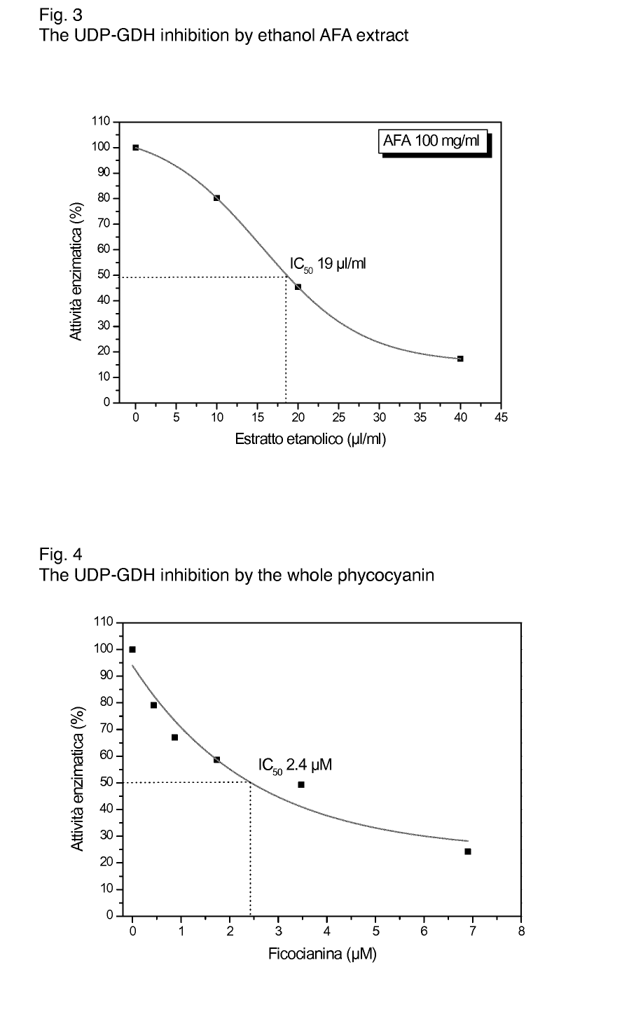

We have tested the ability of the two algal extracts, and found a significant and dose-dependent ability of both to inhibit the enzymatic activity of UDP-GDH, with an IC50 of 18 μ/ml for the water extract (FIG. 2) and 19 μ/ml for the ethanol extract (FIG. 3). We can thus establish a general ability of the whole algae to inhibit UDP-GDH with a IC50 of 18.5 μ/ml. In both cases, the inhibition has appeared to be of a mixed type in relation to both the UDP-GDH enzyme and to the cofactor NAD₊.

We have then tested the UDP-GDH inhibition property of the single purified molecules phycocyanins and of its chromophore phycocyanobilin, obtaining the following results. The phycocyanin has an IC50 of 2.4 μM (FIG. 4) again with an inhibition of a mixed type in relation to both the UDP-GDH enzyme and the cofactor NAD+.

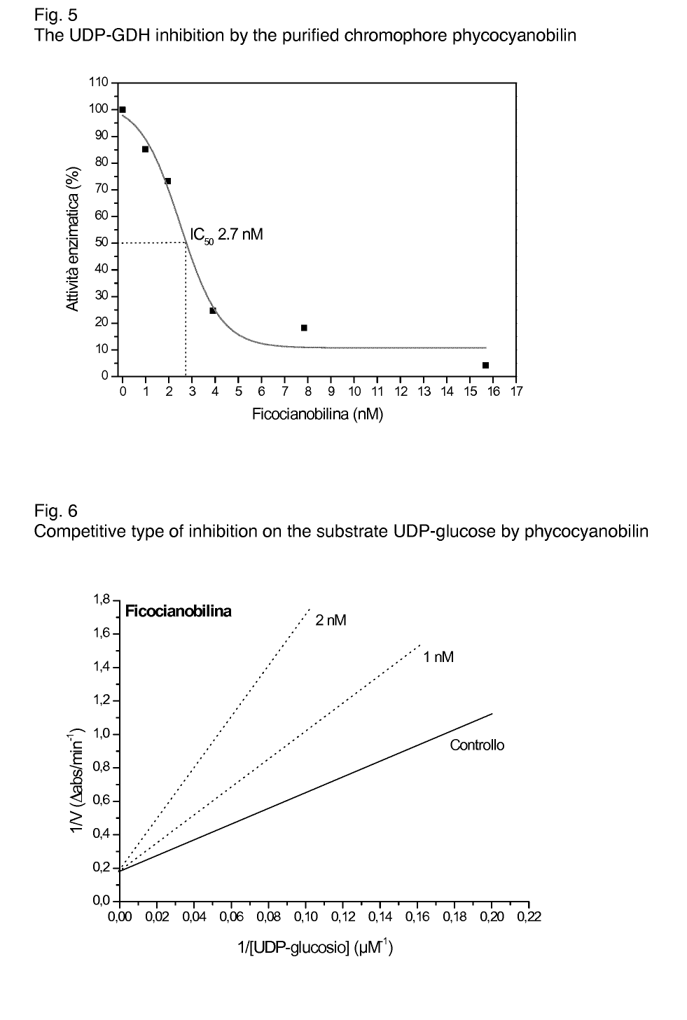

The most remarkable result has been obtained with the pigment phycocyanobilin, whose IC50 has been established at the level of nanomolars, and specifically at 2.7 nM (FIG. 5). Most interesting is the fact that the type of inhibition performed by the PCB is directly competitive towards the UDP-glucose substrate of the UDP-GDH enzyme, thus showing an ability to directly block the attachment of the enzyme to its substrate (FIG. 6).

### 2) In Vivo Human Experimentation on the Ability of AFA Micoalgae to Increase Curcumin Absorption

Methodology

In order to verify our invention, we have tested a formula composed of approximately ⅔ of curcumin and ⅓ of AFA on 5 healthy voluntary subjects, between 25 and 37 years of age. First we had the subjects ingest approx. 4.5 grams of curcumin capsules alone (Turmeric extract at 95% curcumin), and checked the presence of curcumin in the plasma. About a week later, to allow for the elimination of any residue of curcumin in the organism, we had the 5 subjects ingest a total of approx. 3 grams of the same Turmeric extract at 95% curcumin together with 1.5 grams of AFA algae or an AFA extract concentrating its phycocyanins (PC) and phycocyanobilin (PCB).

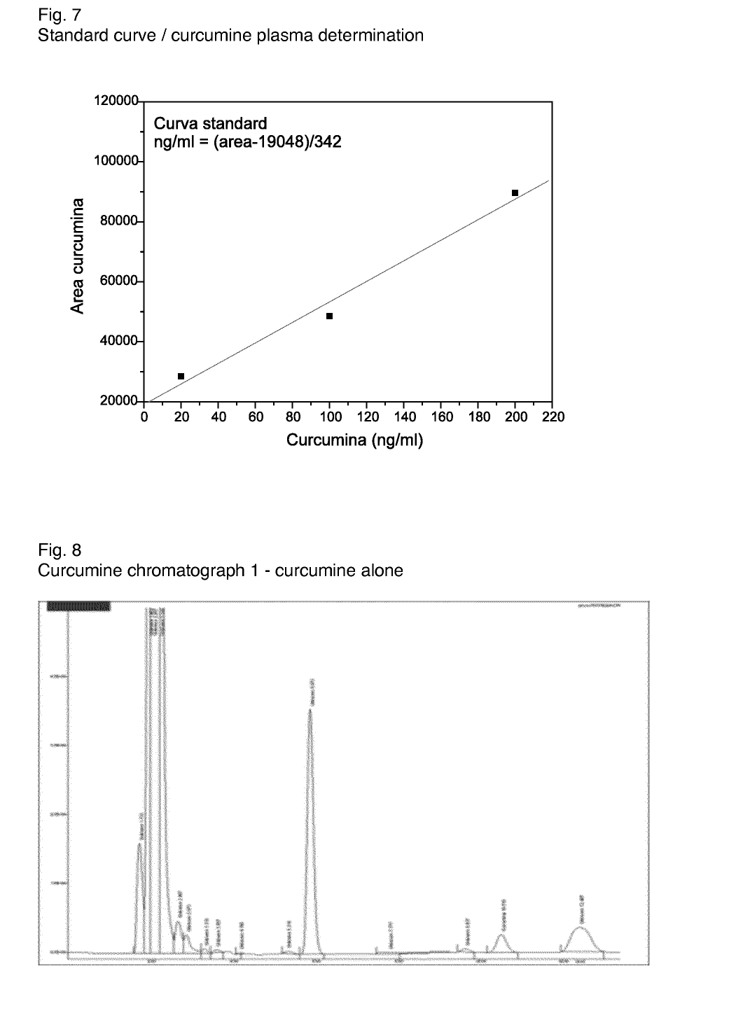

Blood was taken from each subject, through heparinized tubes, 5 times: at T0, after 15 min., 30 min., 60 min. and 120 min. After centrifuging the samples at 2500 rpm for 10 min., samples were conserved at −20° until the time of plasma analytical determination. The latter was performed through a technique, using HPLC to determine curcumin's plasmatic concentration, as previously described by Zengshuan et al. (10). The samples were prepared as follows: 100 μl of plasma have been treated in an Eppendorf tube with the same quantity (100 μl) of acetonitrile (ACN). The sample has been agitated through vortex for 5 min., then centrifuged for 5 min, at 14000 g. The supernatants thus obtained have then been injected into the HPLC. The plasmatic concentration of curcumin (ng/ml) is then calculated with the formula described in FIG. 7.

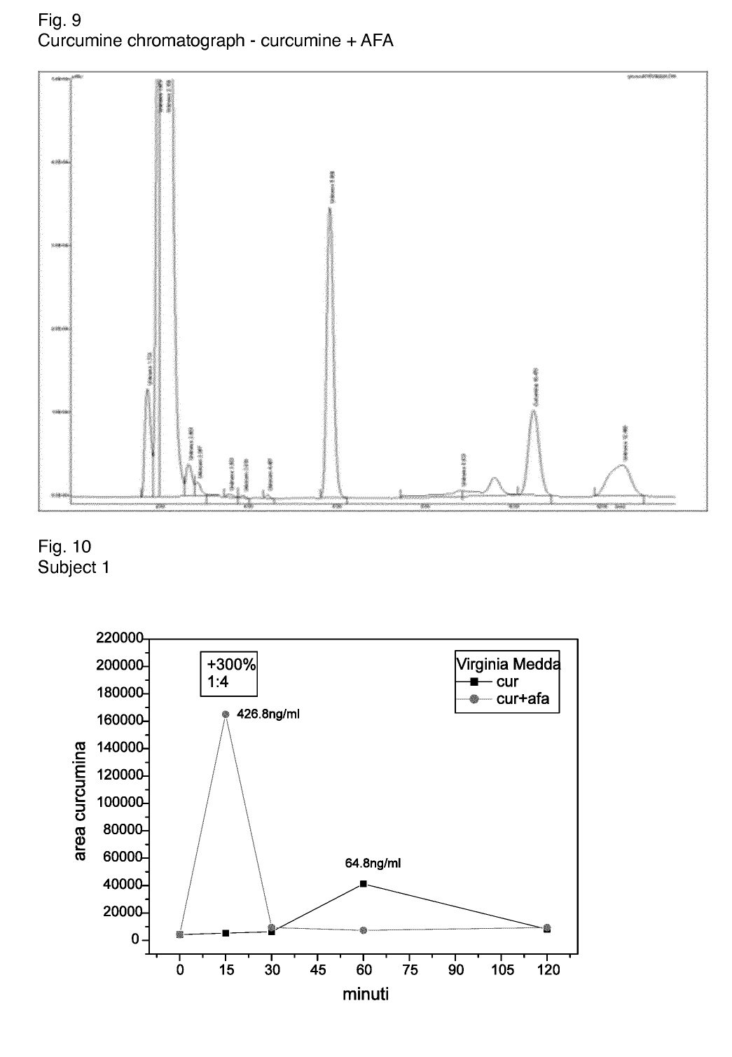

The chromatograms obtained on the two curcumin formulas with the technique described above are reported in FIG. 8 and FIG. 9.

### Results Obtained

Below we report the results obtained in the 5 cases tested. However, we must clarify that the results given in the graphs must then be adjusted to the fact that the quantity of curcumin administered with AFA algae was of 3 grams, whereas the quantity of curcumin administered alone was of 4.5 grams, or 150% more curcumin. To adjust for this we have increased of 50% the results obtained with the AFA+curcumin products.

### A) Curcumin+AFA Algae

Case 1—The subject number 1 had the following results: a) curcumin alone had a maximum plasma absorption of 64.8 ng/ml, with the peak being reached after approximately 1 hour after ingestion; b) the blend of curcumin+AFA had a maximum absorption of 426.8 ng/ml., which constitutes a 665% absolute increase (or 300% in terms of AUC), relative to curcumin alone; the absorption peak was reached at around 15 min. after ingestion; while the complete elimination of curcumin metabolites from plasma was achieved at around 30 min. after ingestion (see FIG. 10). Adjusting for the 50% less curcumin used, we can approximately establish an absolute increase of approx. 1000% (10-fold) and of 450% in terms of AUC.

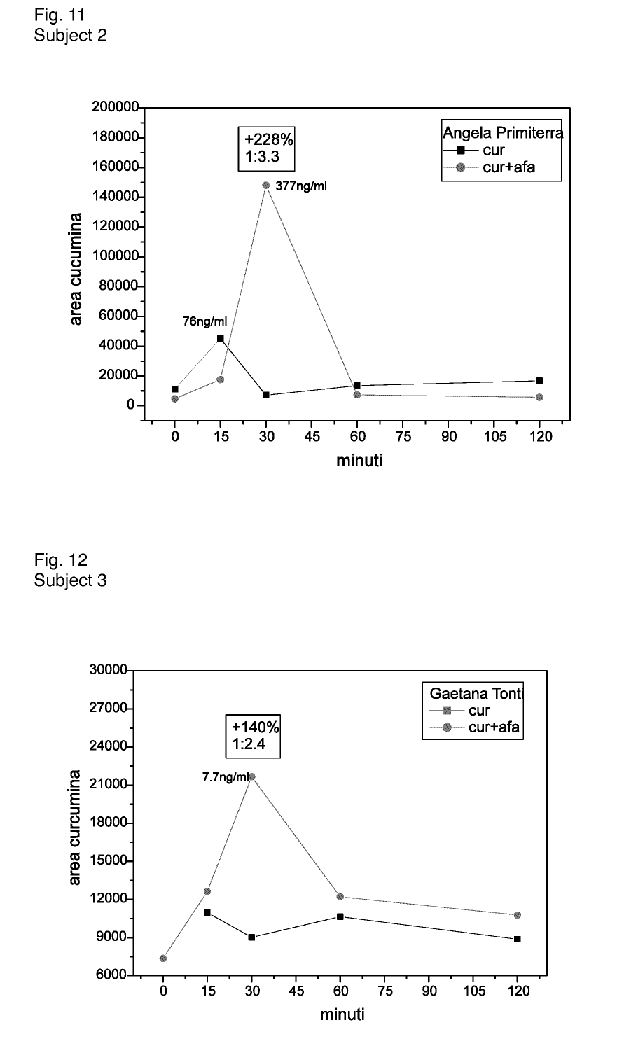

Case 2—The subject number 2 had the following results: a) curcumin alone had a maximum plasma absorption of 76 ng/ml, with the peak being reached after approximately 15 min. after ingestion; b) the blend of curcumin+AFA had a maximum absorption of 377 ng/ml., which constitutes a 228% increase (in terms of standard curve—almost 500% in absolute terms), relative to curcumin alone; the absorption peak was reached at around 30 min. after ingestion; while the complete elimination of curcumin metabolites from the plasma was achieved at around 60 min. after ingestion (FIG. 11). Adjusting for the 50% less curcumin used, we can approximately establish an absolute absorption increase of approx. 750%, and of 340% in terms of AUC.

Case 3—The subject number 3 had the following results: a) curcumin alone had a maximum plasma absorption below weight quantity detection; b) the blend of curcumin+AFA had a maximum absorption of 7.7 ng/ml., which constitutes a 140% increase relative to curcumin alone (the absolute comparison is not possible here for lack of weight quantification of curcumin alone); the absorption peak was reached at around 30 min. after ingestion; while the elimination of the major percentage of curcumin metabolites from the plasma was achieved at around 60 min. after ingestion, with a further slow reduction in the 2nd hour (FIG. 12). Adjusting for the 50% less curcumin used in the mix formula, we can approximately establish an increase of approx. 220% in terms of AUC.

### B) Curcumin+AFA PC Concentrate

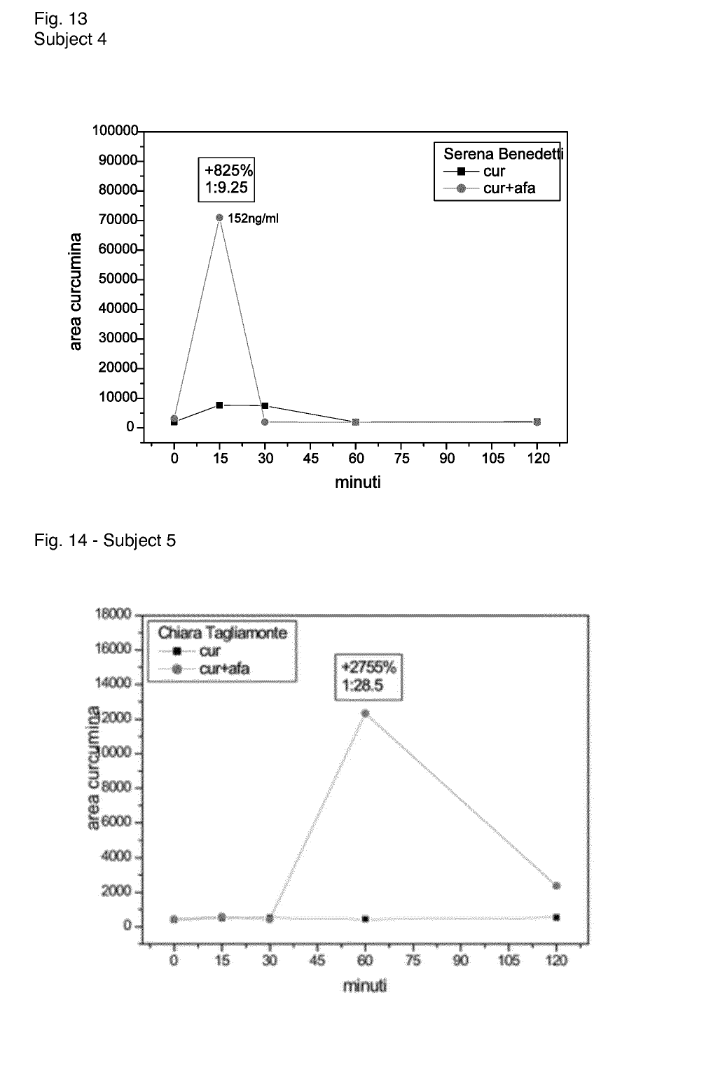

Case 4—The subject number 4 had the following results: a) curcumin alone had a maximum plasma absorption below weight quantity detection; b) the blend of curcumin+AFA algae had a maximum absorption of 152 ng/ml., which constitutes a 825% AUC increase relative to curcumin alone (the absolute comparison is not possible here for lack of weight quantification of curcumin alone); the absorption peak was reached at around 15 min. after ingestion; while the complete elimination of curcumin metabolites from the plasma was achieved at around 30 min. after ingestion (FIG. 13). Adjusting for the 50% less curcumine used, we can approximately establish an absorption increase of approx. 1235% in terms of AUC.

Case 5—Case 5 was actually tested through a slightly different methodology, using a different technique to determine the curcumin concentration in plasma, a technique previously described by Ji Li et al. (10). 200 μl of plasma sample was treated in an Eppendorf tube with the same quantity (200 μl) of acetonitrile (ACN). The resulting material has been agitated by vortex for 5 min., then centrifuged for 5 min. at 2500 g. The supernatant thus obtained was then injected in HPLC. In this case, and with this methodology, the comparison was done only by the AUC difference, without any reference for neither samples to weigh quantity. The result obtained for Subject 5 was the following: the blend of curcumin+AFA extract had a maximum absorption 28.5 times higher than curcumin alone, which represents a staggering 2755% increase in plasma curcumin absorption. The absorption peak was reached at around 1 hour after ingestion; while the almost complete elimination of curcumin metabolites from the plasma was achieved at around 120 min. after ingestion (FIG. 14). Adjusting for the 50% less curcumin used, we can approximately establish an absorption increase of approx. 4130% (40-fold) in terms of AUC.

C) A further Case: Spirulina+Curcumin Formula

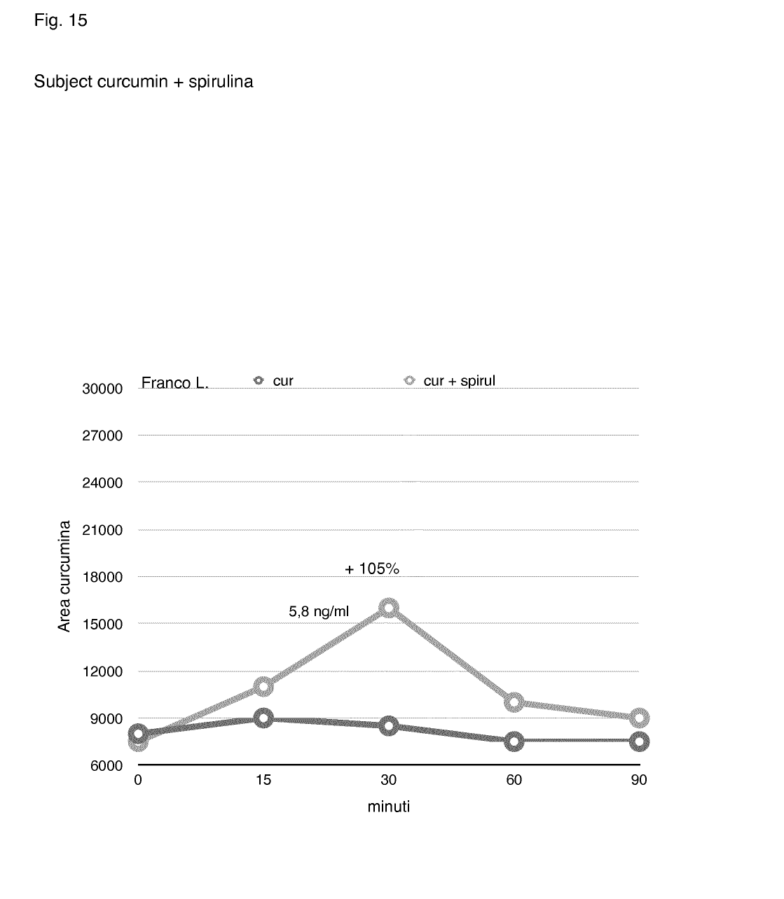

In order also to evaluate if similar results could be obtained by mixing curcumin with other microalgae containing phycocyanin and phycocyanobilin, we tested another subject by following the administration and evaluation methodology outlined above, but mixing 3 grs. of curcumin with 1.5 grs. of Spirulina. The result obtained was also positive, with an increase in curcumin absorption of +105% (FIG. 15), less than what found with AFA, but still significant, and clearly due to the fact that Spirulina too, like all microalgae, contain phycocyanin, but most of all they contain the pigment phycocyanobilin, which we have seen is the most powerful and specific UDP-GDH inhibitor.

Considerations on Efficacy and Comparison with other Substances.

Looking at the results obtained the 5 human subjects tested some important elements emerge, both in relation to the difference between AFA algae and its concentrate, as well as Spirulina, and in relation to other substance used to increase curcumin absorption, such as pipeline and phosphatidylcholine.

Plasma Absorption: AFA Algae vs. Phycocyanins Concentrate.

The first thing to remark is the high level of increased absorption generated by adding AFA algae to curcumin; in the 3 cases tested, the increase was respectively of 450%, 340% and 220%. These are significant results, though inferior, as it was to be expected, to the absorption increases produced by the AFA extract, which were respectively of 1235% and 4130%! These difference make it plausible to think of a different use of the two AFA substances: the whole cyanobacterial AFA algae, also in consideration of its lower cost of production, could be used for preventative and health maintainance purposes; whereas the phycocyanin concentrate could be reserved for more specifically therapeutic uses. The same applies to Spirulina: its lower performance makes it potentially less useful for therapeutic usages; and even though in general the use of AFA algae is preferred, the very low cost of Spirulina and its wider availability, makes it a good candidate to increase curcumin absorption for preventative and health maintenance purposes.

Plasma Absorption: AFA and AFA Extract vs. other Absorption Enhancers.

If we compare the microalgal substances with the increased absorption obtained by the use of other substances, we see the following:

a) Vs. Piperine. Piperine generated in animals a plasma concentration increase of 154%; while in humans the increase was 2000%, but only because in absolute terms, and most of all because the baseline of comparison, curcumin alone, was at the non detectable level (11). Piperine is extracted from black pepper, and in our cases 4 and 5, where also an AFA extract was used, the increases in curcuminoids absorption was respectively +1235% and +4130%, thus on average the absorption increase was significantly higher than that generated by piperine, but with the added advantage that, as opposed to piperine, AFA does not pose any side effects problems or health risks.

b) Vs. phosphatidylcholine.

Another way of improving the bioavailability of curcumin is that of mixing it with phosphatidylcholine (Meriva®). Various studies have shown that the plasma levels of curcumine are significantly improved when curcumin is mixed with phosphatidylcholine. For instance, in a recent study on absorption, it was shown that Meriva® generated a 29-fold increase in the concentration of curcumin metabolites relative to curcumine alone (12). However, in this study only curcumine metabolites were found, and so it is not really comparable with our test, also given that churchman's metabolites are not as powerful as curcumin itself. Another study showed that the absorption of curcumin was increased 5-fold when it was complexed with phosphatidylcholine (7). Compared to this, AFA algae generated a 3-4-fold absorption increase, which is slightly lower than Meriva®, yet with the added advantage that AFA algae provides also a vast array of synergistic anti-oxidant and anti-inflammatory factors, which make the blended product much more effective health-wise. As to the AFA extract, it is clearly much superior, as it generates up to a 40-fold increase in curcumin absorption, besides providing an even more concentrated set of anti-oxidant, anti-inflammatory and anti-cancer molecules which add up to the very same properties from curcumin.

c) Vs. Curcumin nanoparticles. A recent study compared the plasma absorption of uncomplexed curcumin, curcumin+piperine and a new delivery system based on curcumin nanoparticles (13). The data and results were the following:

| | Curcumin dose | Cmax | AUC |
|---|---|---|---|
| Formulation | (mg/kg) | (ng/ml) | (ng/ml h) |
| Curcumin NPs | 100 mg | 260.5 | 3224 |
| Curcumin + 10 mg. Piperine | 250 mg. | 121.2 | 872 |
| Curcumine | 250 mg. | 90.3 | 312 |

From this table we can draw the following conclusions. First, the absolute dose of curcumin in a human being of 60 kgs. would be of 15 gr. for the pure curcumin and the curcumine+piperine, and of 6 gr. for the curcumin nanoparticles, which in this respect seem to be significantly more effective than piperine in terms of absorption increase (even though piperine has the advantage of having health properties of its own that can be added to those of curcumin). If we compare these results with those of AFA+curcumin, or even better with those of curcumin+AFA extract, we see the following:
A) in relation to piperine, looking at the respective AUC absorption increase, adjusted for the significant dose difference (3 gr. of curcumin with AFA vs. 15 gr with piperine, or 5 times less), the AFA+curcumine product is on average 200% more powerful than piperine; whereas, when we compare the AFA-extract AUC increase absorption with that of piperine, again adjusted for the significantly different dose, we find that the AFA-extract is 15-times, or 1500%, more powerful than piperine.
B) In relation to the Curcumin nanoparticles (NPs), the comparison between this and the AFA extract, adjusted for the dose difference, gives that the AFA extract is still 163% more effective than the curcumine NPs in terms of absorption enhancement. Of course, we must also consider that NPs add nothing in terms of health benefits, except the increased absorption of curcumine; whereas the AFA extract has health properties at least as significant, in terms of antioxidant and antinflammatory activity, as those of curcumin itself.

### Some Final Considerations

Apart from the comparison with other substances, what can we say about the efficiency and efficacy of the curcumin+cyanobacterial algae and/or extract blend? Though more specific pharmacokinetic studies will be needed, we can already draw some interesting preliminary conclusions. If we look at the timing of curcumin absorption, we can see that in 2 cases the absorption peak happens after 15 min. from ingestion, and curcumin is eliminated from the plasma after just 30 minutes; in 2 more cases the peak is at 30 minutes and plasma clearance after 1 hour; and finally in the last case the peak is after 1 hour and clearance is completed after 2 hours. In general, these times are faster than those usually applied to drugs, which on average reach their plasma peak concentration at about 1 hr. after ingestion, and then may take up to 10 hours to be cleared from the blood through the excretion path. This may mean only one thing, namely that the passage of curcumin through the gastrointestinal barrier, thanks to its synergy with the cyanobacterial algae, is very fast, and so very efficient. The process of glucoronidation happens at this level, and this is the reason why, when curcumin alone or with non efficient co-factors are ingested, in the blood one finds much more glucoronidated metabolites than curcumine itself. The fact that with the microalgae and/or extract there is a significant and very rapid increase of the whole curcumin in the blood means that the intestinal glucoronidation process is significantly prevented, and curcumine is absorbed in the blood for the most part as such. This is already an excellent result. The next phase is the permanence of the curcumin in the blood, which is also very short. Now, we have no elements at this point to evaluate what happens to the curcumin when it leaves the blood, and only further pharmacokinetic studies can clarify that. However, the fact that the curcumin in the blood exits the blood very fast it can only mean that curcumin is transferred from the blood into organs and tissues, and even if a certain percentage may be then eliminated by the kidneys, it is likely that a large part does actually moves into the most blood-supplied organs such as the heart and the liver. In general, it looks that the rapidity of action of the blend curcumin-algae is a manifestation of its high efficacy.

### NOTES

1) Sharma O P., Antioxidant activity of curcumin and related compounds, in Biochem Pharmacol 1976; 25:1811-1812.
2) Surh Y J, Chun K S, Cha H H, et al., Molecular mechanisms underlying chemopreventive activities of anti-inflammatory phytochemicals: down-regulation of COX-2 and iNOS through suppression of NFkappa B activation, in Mutat Res 2001; 480-481:243-268.
3) Aggarwal B B, Kumar A, Bharti A C., Anticancer potential of curcumin: preclinical and clinical studies, in Anticancer Res 2003; 23:363-398.
4) Jurenka J., Anti-inflammatory Properties of Curcumin, a Major Constituent of Curcuma longa: A Review of Preclinical and Clinical Research, in Alternative Medicine Review Volume 14 (2), 2009, pp. 141-53.
5) Ireson C, Orr S, Jones D J, et al., Characterization of metabolites of the chemopreventive agent curcumin in human and rat hepatocytes and in the rat in vivo, and evaluation of their ability to inhibit phorbol ester-induced prostaglandin E2 production, in Cancer Res 2001; 61:1058-1064.
6) Shoba G, Joy D, Joseph T, et al. Influence of piperine on the pharmacokinetics of curcumin in animals and human volunteers, in Planta Med 1998; 64:353-356.
7) Marczylo T H, Verschoyle R D, Cooke D N, et al. Comparison of systemic availability of curcumin with that of curcumin formulated with phosphatidylcholine, in Cancer Chemother Pharmacol, 2007; 60:171-177.
8) Huh, J. W., Choi, M. M., Yang, S. J., Yoon, S. Y., Choi, S. Y. and Cho, S. W. (2005) Inhibition of human UDP-glucose dehydrogenase expression using siRNA expression vector in breast cancer cells. Biotechnol. Lett. 27, 1229-1232.
9) Benedetti S., Scoglio S. et al., Oxygen Radical Absorbance Capacity of Phycocyanin and Phycocyanobilin from the Food Supplement Aphanizomenon flos-aquae, in J Med Food 13 (1) 2010, 1-5.
10) Zengshuan Ma, Anooshirvan Shayeganpour, Dion R. Brocks, Afsaneh Lavasanifar and John Samuel (2007), High-performance liquid chromatography analysis of curcumin in rat plasma: application to pharmacokinetics of polymeric micellar formulation of curcumin, in Biomedical Chromatography, 21:546-552.
11) Shoba G. et al., Influence of piperine on the pharmacokinetics of curcumin in animals and human volunteers, in Planta Med. 1998 May; 64(4):353-6.
12) Cuomo J. e al., Comparative absorption of a standardized curcuminoid mixture and its lecithin formulation, J Nat Prod. 2011 Apr. 25; 74(4):664-9. Epub 2011 Mar. 17.
13) Shaikh J. et al, Nanoparticle encapsulation improves oral bioavailability of curcumin by at least 9-fold when compared to curcumin administered with piperine as absorption enhancer, Eur J Pharm Sci. 2009 Jun. 28; 37(3-4):223-30.

1. A composition of cyanobacterial algae and cyanobacterial algae extracts, containing phycocyanins and phycocyanobilins blended with curcumin in varying proportions, to enhance the absorption of curcumin, to inhibit the enzyme UDP-GDH.

2. (canceled)

3. The composition according to claim 1, whereby the preferred microalgae used is of the species Aphanizomenon flos aquae.

4. The composition according to claim 1, whereby the microalgae used is of the species Spirulina.

5. The composition according to claim 1, whereby the microalgae is any cyanobacterial microalgae or microalga extract containing phycocyanins and phycocyanobilins.

6. (canceled)

7. (canceled)

8. The composition according to claim 1, whereby said compositions are prepared in a pharmaceutically acceptable form.

9. The use of the composition according to claim 8 in which the subject of the composition's administration is a human being to enhance the absorption of curcumin to inhibit the enzyme UDP-GDH.

10. The use of the composition according to claim 8 in which the subject of the composition's administration is an animal to enhance the absorption of curcumin to inhibit the enzyme UDP-GDH.
